(19) United States
(12) Patent Application Publication
(10) Pub. No.: US 2013/0345782 A1
(43) Pub. Date: Dec. 26, 2013

(54) ELECTRODE FOR SUBCUTANEOUS ELECTOLIPOLYSIS

(71) Applicant: Ambrosios Minas Kambouris, Mildura (AU)

(72) Inventor: Ambrosios Minas Kambouris, Mildura (AU)

(21) Appl. No.: 13/982,311

(22) PCT Filed: Jan. 30, 2012
(86) PCT No.: PCT/AU12/00065

§ 371 Date: Jul. 30, 2013

(30) Foreign Application Priority Data
Jan. 28, 2011 (AU) 2011100113
Feb. 21, 2011 (AU) 2011900578

(51) Int. Cl. A61N 1/05

(52) U.S. Cl. CPC A61N 1/0504
USPC 607/116

(57) ABSTRACT

An electrode or conductive device for subcutaneous electrolipolysis allows long electrolipolysis treatment times whilst the patient is fully ambulant. This effectively revolutionises such treatment for use in localised fat deposit reduction. The conductive device for delivering an electrical current into subcutaneous tissue includes a stem and two or more invasive electrodes configured to allow for independent articulation between the stem and one of the two or more invasive electrodes and/or between the two invasive electrodes.

### FIELD OF THE INVENTION

This invention relates to subcutaneous electrolipolysis that allows long electrolipolysis treatment times whilst the patient is fully ambulant.

### BACKGROUND TO THE INVENTION

Lipolysis is a natural biochemical process by which triglyceride stored in animal or human fat cells is reduced to glycerol and free fatty acids, with a consequential reduction of fat cell volume. It can be stimulated either by cold temperature (cryolipolysis) or by applying electrical currents through the subcutaneous fat cell layer (electrolipolysis).

Medically, electrolipolysis is carried out using acupuncture needles as electrodes. These are placed into the subcutaneous fat layer parallel to the skin and a pulsed electric current is then applied through them. Needles, rather than pads are used for electrolipolysis due to the inability of surface pads to deliver enough concentrated current around the fat cells to stimulate lipolysis.

The drawbacks of using acupuncture needles for electrolipolysis include the requirement of a Medical practitioner with experience in needle insertion, patient discomfort, pain, bruising and patient compliance. The development of a multiple needle electrode has overcome some of these drawbacks. These include, reducing pain and technical difficulty related to needle insertion and obtaining optimum penetration depth into the subcutaneous fat layer.

The Multiple Needle Electrode (MNE) is a long flexible electrode with a conductive stem and multiple fine needles protruding perpendicularly from this stem. These fine needles are then pressed into the skin before attaching the conductive stem supporting the needles to a pulsed electric generator (medical device). This electrode has similar discharging effectiveness as the acupuncture needles, demonstrated with subcutaneous voltage maps across similar spaced oppositely charged electrodes implanted in a woman's abdomen.

Commercial application of electrolipolysis involves one hour treatments on a weekly basis for six weeks, and has shown to be effective in reducing the appearance of cellulite and anthropometric measurements in over 100,000 women. This treatment time, using histological and cytological evaluation, is effective in reducing fat cell volume and biochemical changes consistent with an increase in lipolysis.

Further research has demonstrated that extension of the treatment time for electrolipolysis, results in a proportional increase in lipolysis and fat cell volume reduction. These studies conducted by Monash University, using the abdominal fat layer of the Wistar rat, showed that 3 or 6 hours of continuous electrolipolysis, resulted in a cytological reduction of fat cell volume by 11% and 25% respectively.

The ability to quantitatively increase lipolysis by extending electrolipolysis treatment time offers new use possibilities, particularly in reducing localised fat deposits. However, existing electrode technology does not allow for economical or patient compliant treatment options beyond one hour.

The object of this invention is to provide a device that alleviates at least some of the abovementioned problems or provides the public with a useful alternative.

### SUMMARY OF THE INVENTION

Therefore in one form of the invention there is proposed a conductive device for delivering an electrical current into subcutaneous tissue comprising a stem and two or more invasive electrodes configured to allow independent articulation between the stem and one of the two or more invasive electrodes and/or independent articulation between the two or more invasive electrodes.

In preference the stem is conductive.

In preference the two or more invasive electrodes comprise insulation at the point of contact with the skin.

In a further form of the invention there is proposed a method for delivering an electrical current to subject in need thereof, the method comprising the step of applying a conductive device according to any one of claims 1 to 3 to the subject.

In preference the method optionally comprises the step of using an adhesive to hold the conductive device in place on the skin to allow ambulation in the subject.

In preference the electrical current is delivered for a period of more than 1 hour.

### BRIEF DESCRIPTION OF THE DRAWINGS

The accompanying drawings, which are incorporated in and constitute a part of this specification, illustrate various implementations of the invention and, together with the description, serve to explain the advantages and principles of the invention. In the drawings:

FIG. 1 illustrates a first embodiment of the invention with a single row of pins powered by a power source;

FIG. 2 illustrates an embodiment as in FIG. 1 having two rows of pins and supported between two sets of top and bottom layers;

FIG. 3 illustrates an embodiment as in FIG. 2 having two rows of pins both rows supported between a single top and bottom layer;

FIG. 4 illustrates an embodiment as in FIG. 1 having no bottom layer but rather a template;

FIG. 5 illustrates an embodiment as in FIG. 4 but with two templates;

FIG. 6 illustrates an embodiment as in FIG. 5 with two rows of pins both located by using a single template;

FIG. 7 illustrates an embodiment of the invention for a single row of pins using a conductive flexible cloth tape;

FIG. 8 illustrates an embodiment as in FIG. 7 but with two rows of pins;

FIG. 9 illustrates an embodiment as in FIG. 8 with one bottom plate;

FIG. 10 illustrates an embodiment having insertable pins mounted in a flexible conductive pad;

FIG. 11 is a cross-sectional view of the embodiment of FIG. 10;

FIG. 12 illustrates an embodiment as in FIG. 10 with a different pattern for the pad;

FIG. 13 is an embodiment as in FIG. 10 but with two rows of pins supported by a single pad;

FIG. 14 illustrates yet another embodiment of mounted pins;

FIG. 15 is a top view illustrating possible layout of electrodes; and

FIG. 16 is yet another embodiment illustrating the different geometrical arrangement of pins.

### DETAILED DESCRIPTION OF PREFERRED EMBODIMENT

The following detailed description of the invention refers to the accompanying drawings. Wherever possible, the same reference numbers will be used throughout the drawings and the following description to refer to the same and like parts. Dimensions of certain parts shown in the drawings may have been modified and/or exaggerated for the purposes of clarity or illustration.

Turning now t the drawings in details there are various drawings showing different arrangements of pins. What is common between all of the drawings is that there is a power source 01, and a layer of material 02 be it a pad, bandage or the like that covers a set of pins arranged in electrical series by in some cases wire 04 and in others by a conductive pad and joined together in a flexible arrangement. In some cases the pins may extend through a gauze or other pre-cut or pre-marked tape 05.

Rather than going into detail about each of the embodiment, that the reader will clearly understand it is now the intention to generally describe each embodiment without specific reference numbers.

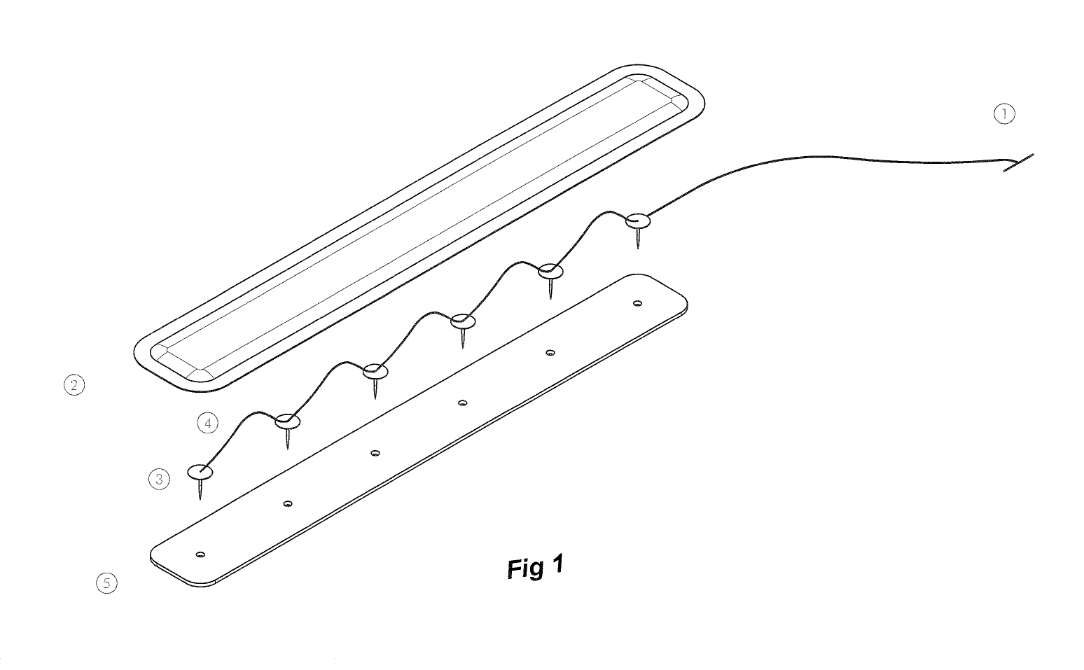

Thus turning to FIG. 1 there is illustrated a set of pins electrically connected in series by a wire and having bottom flexible gauze or other tape that includes apertures for the pins to pass there through, the pins then covered by a bandage, strapping tape or silicon pad.

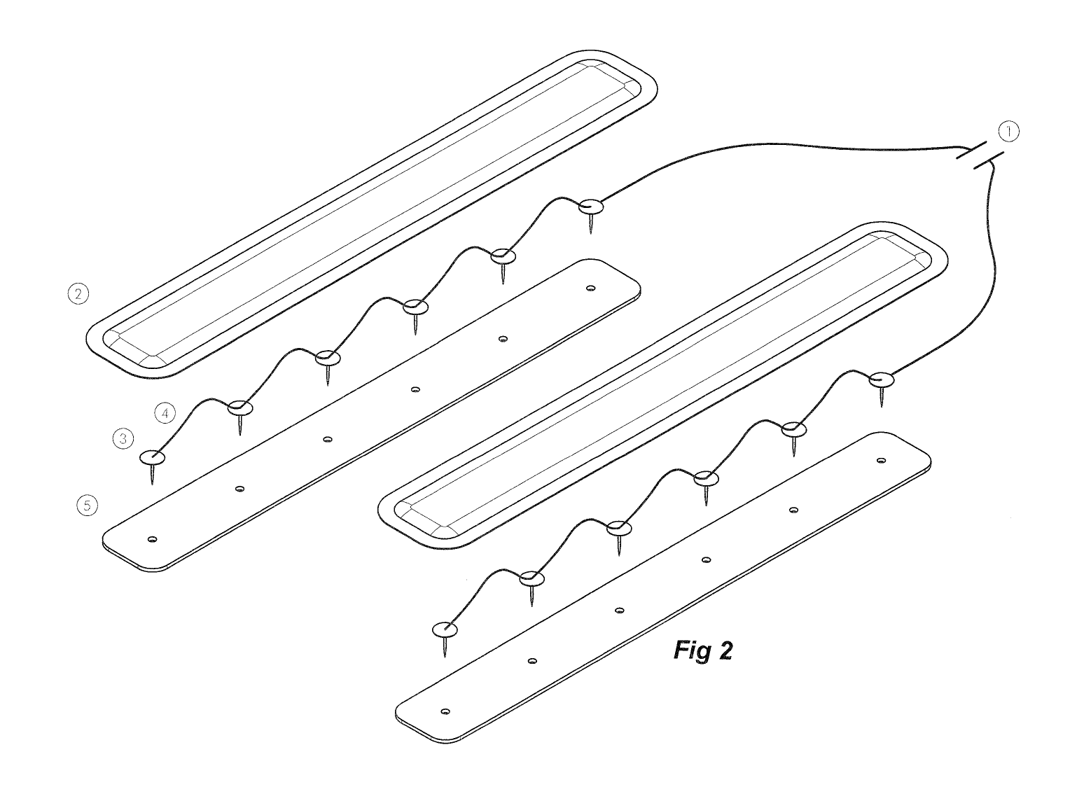

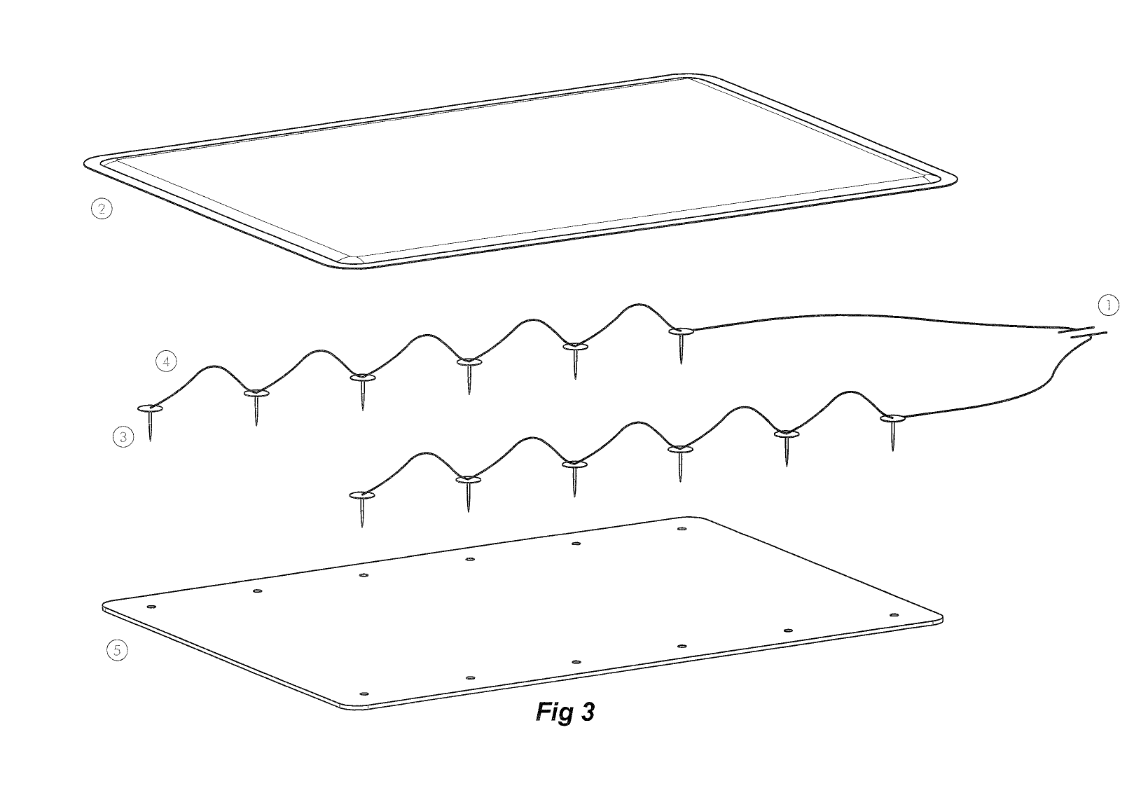

FIG. 2 illustrates the same arrangement but there being two such sets of pins. FIG. 3 then simply illustrates that rather than there being two bottom flexible gauzes there indeed may only be one with a pre-determined spacing.

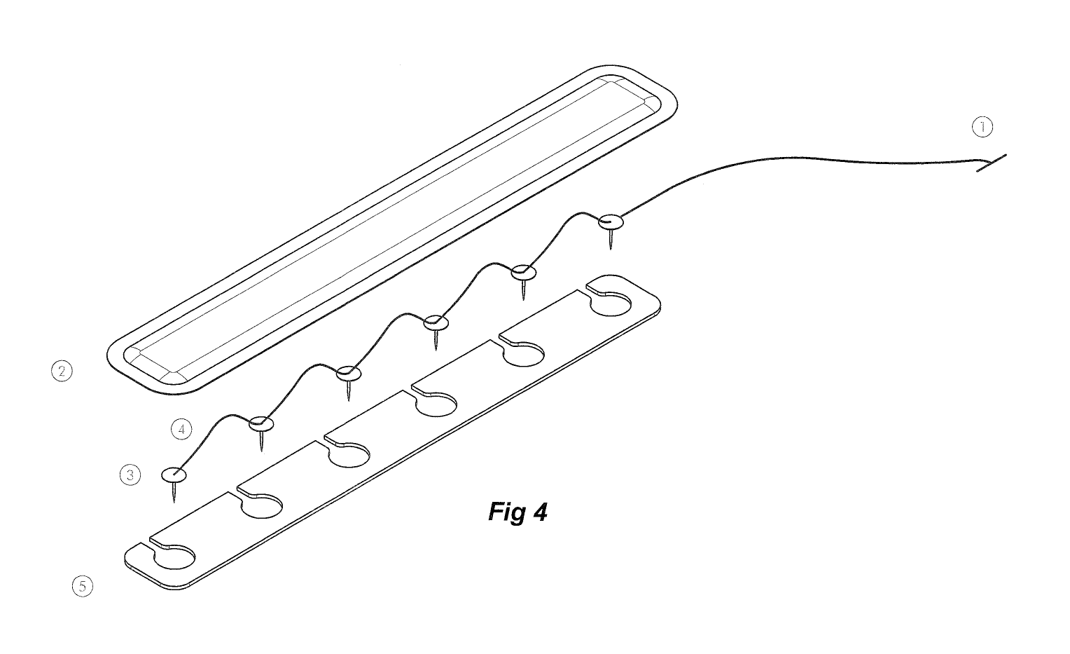

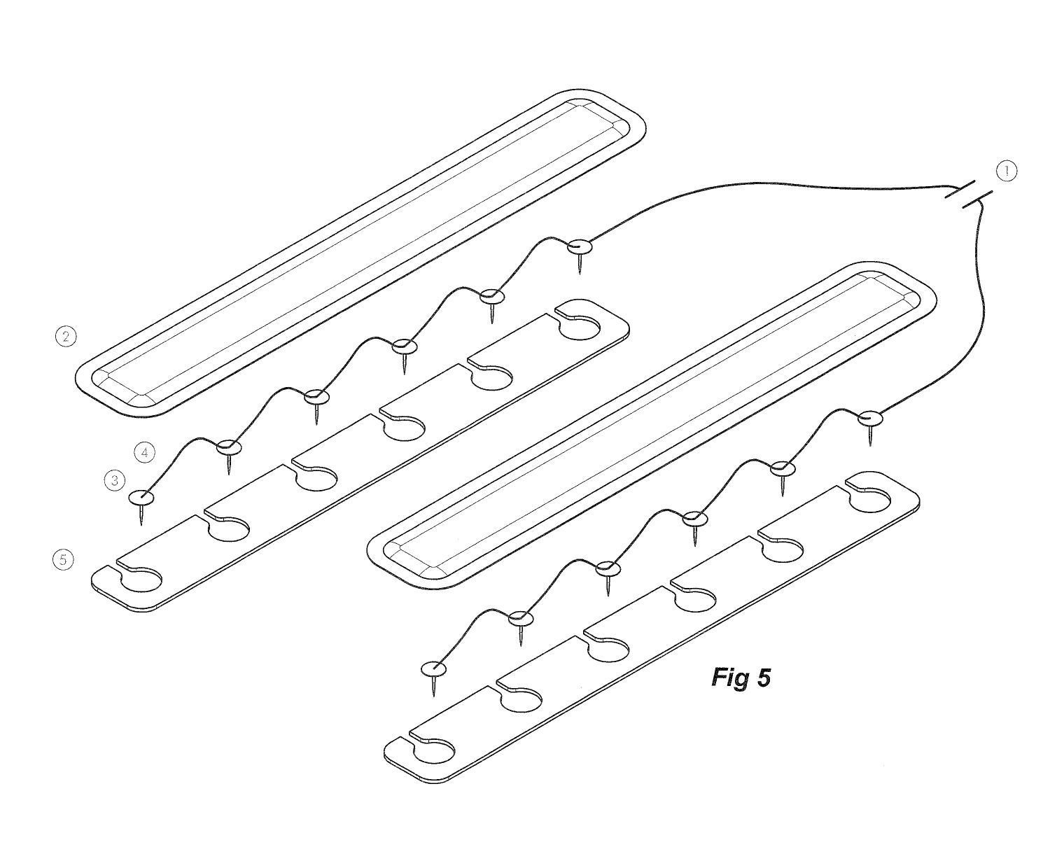

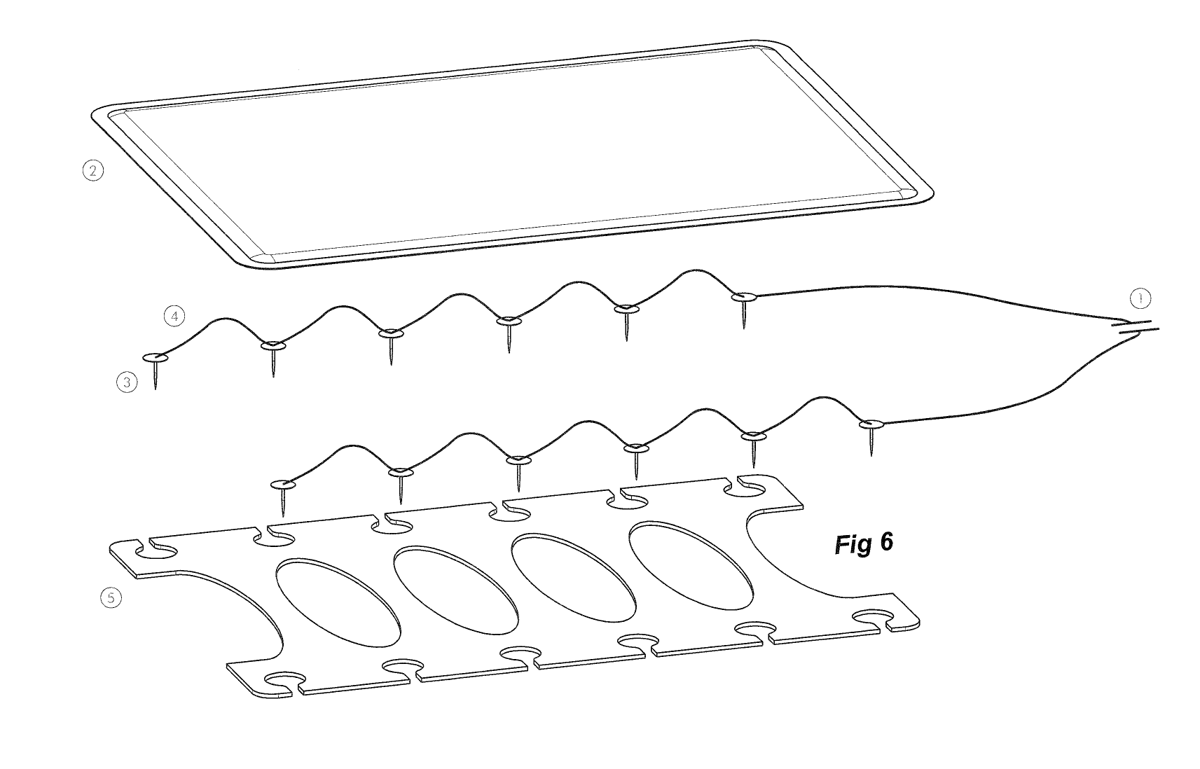

Instead of having a bottom layer, in order to insert the pins into the skin there may be provided a bottom template that is removable once the pins have been inserted and may be made from silicone or be a rigid panel. This is shown in FIG. 4 for a single row, FIG. 5 for two rows and FIG. 6 where there is one template for two rows of pins.

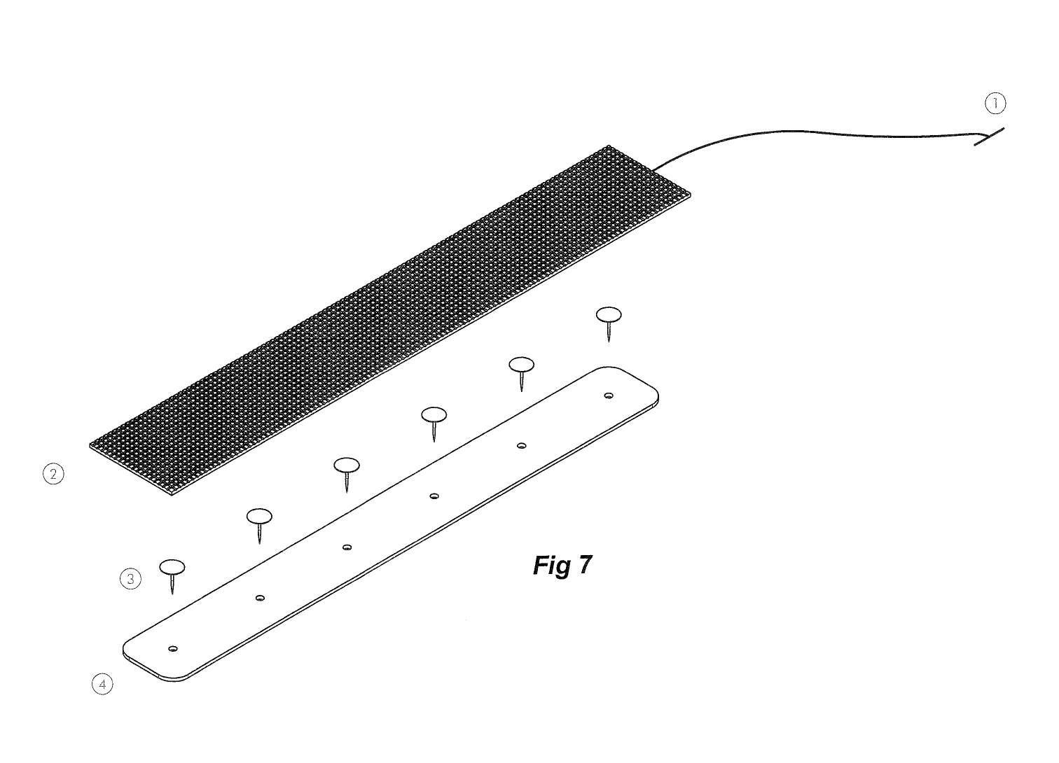

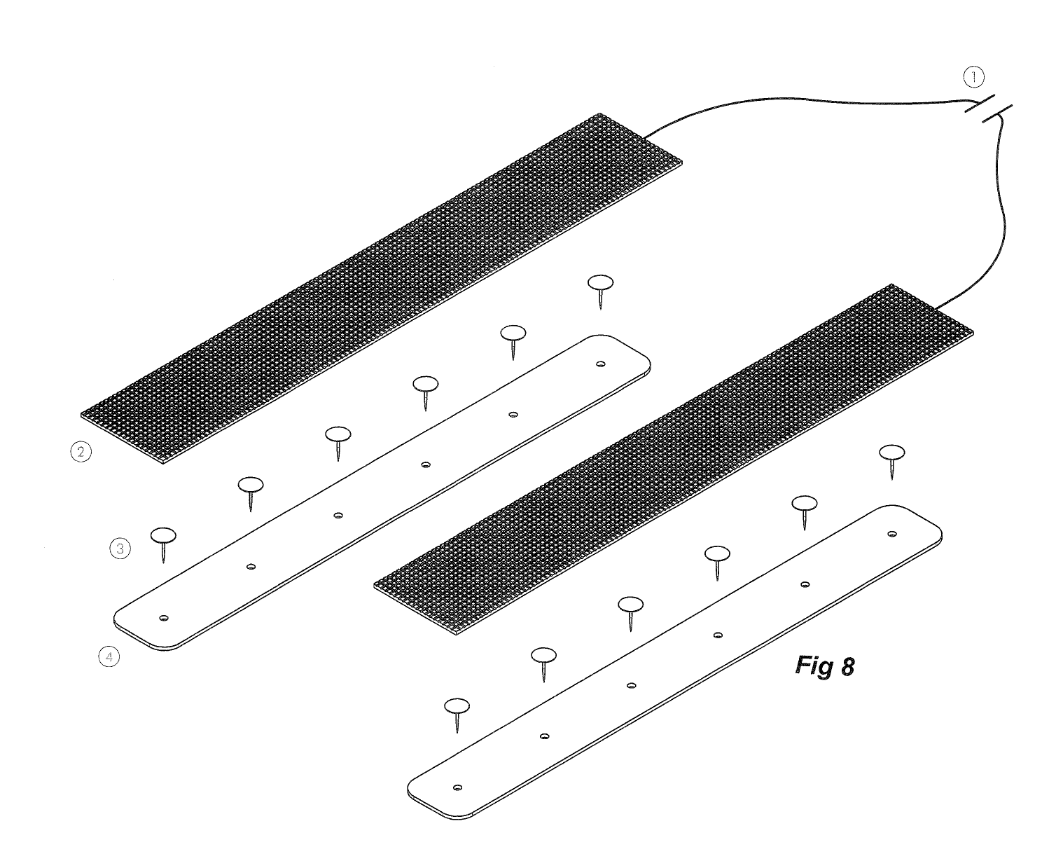

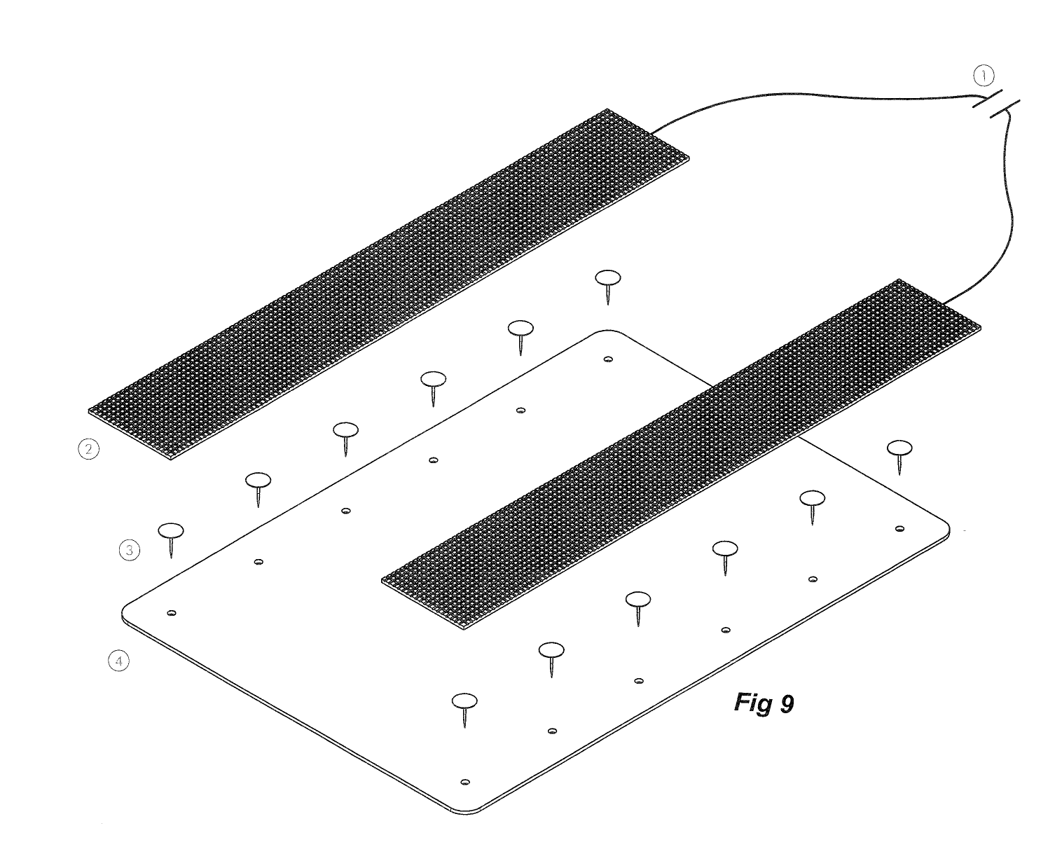

FIG. 7 illustrates an embodiment where instead of the pins connected by an electrical wire the top layer is in fact a conductive flexible cloth tape. FIG. 8 illustrates two rows of pins whilst FIG. 9 shows a single bottom layer for guiding both rows.

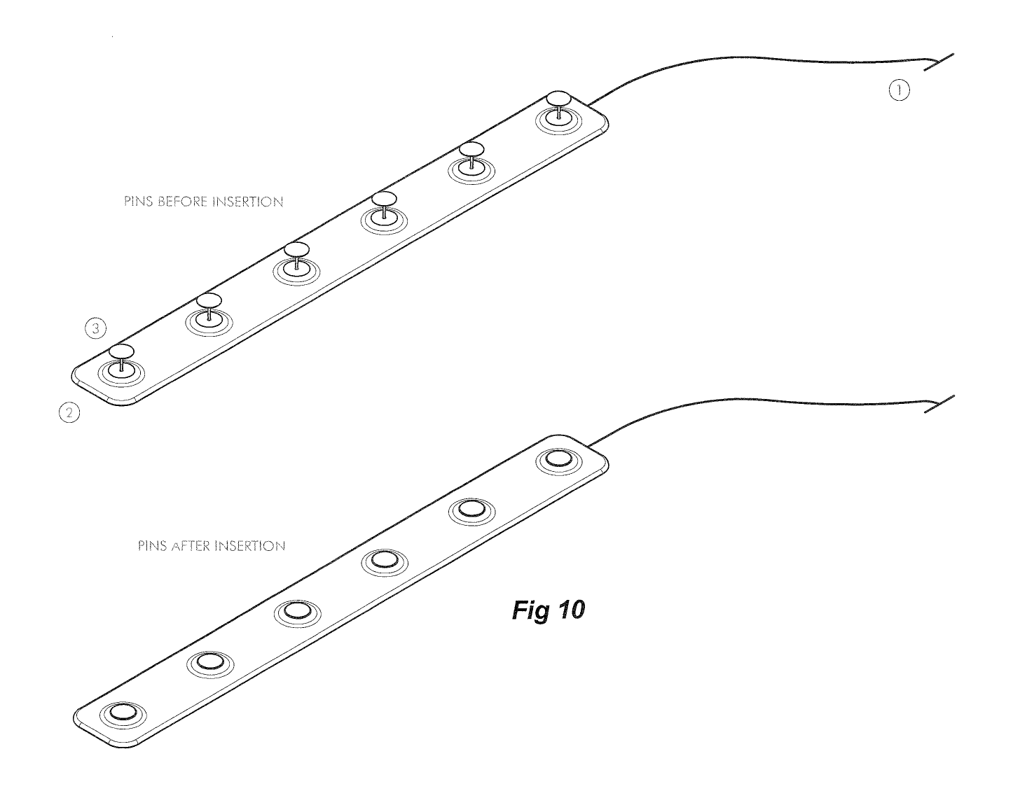

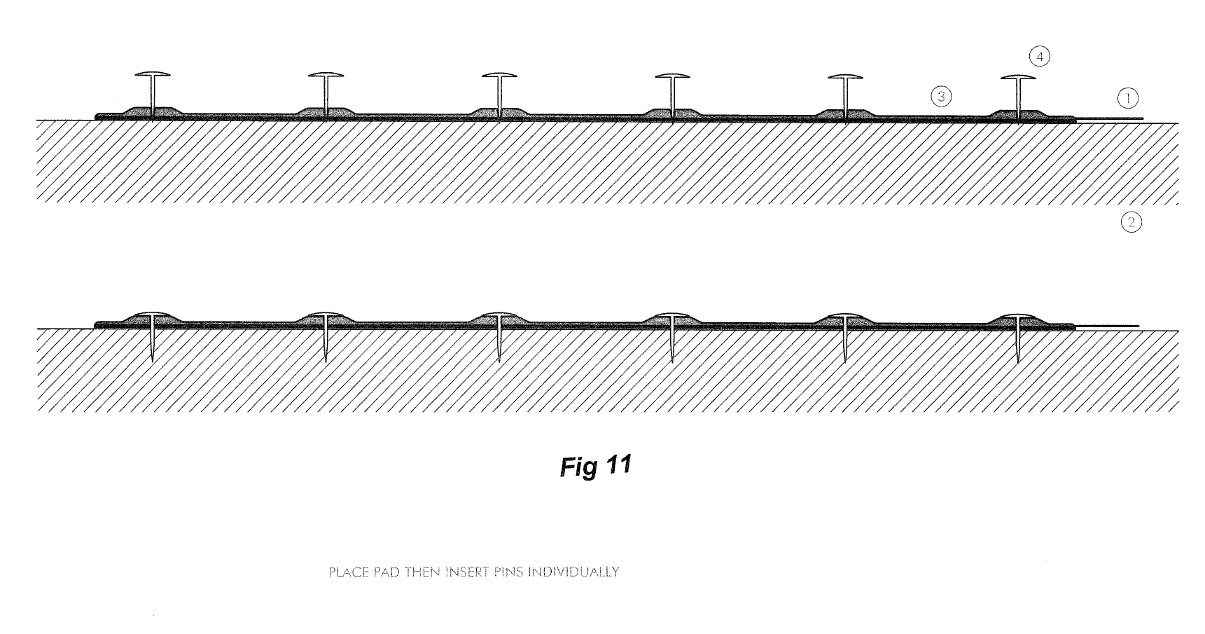

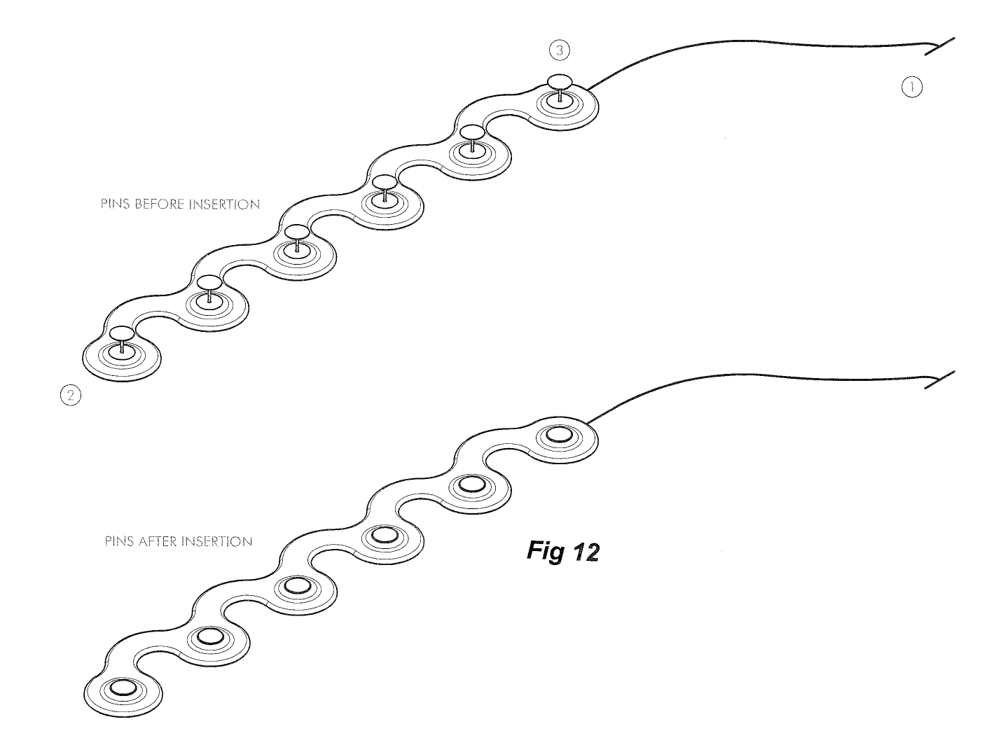

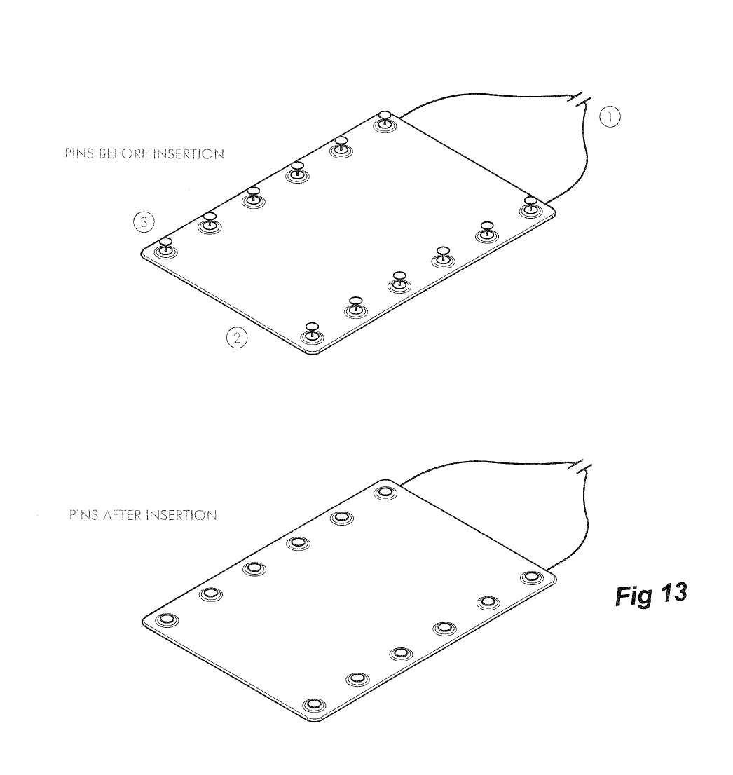

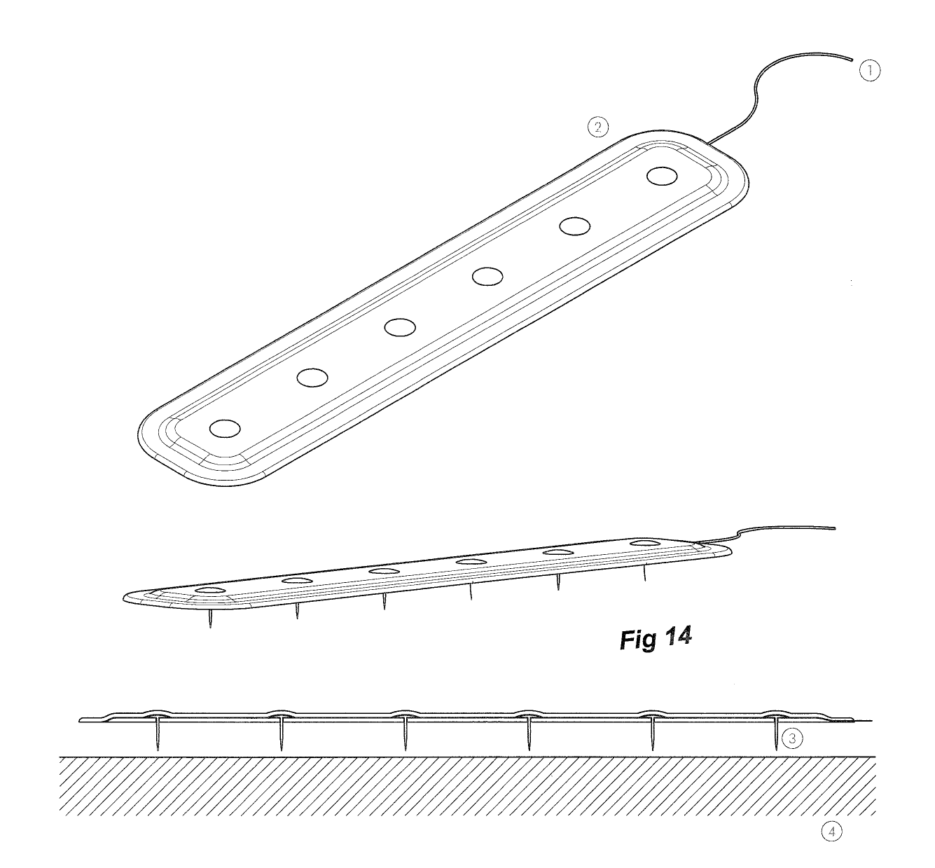

FIG. 10 illustrates an embodiment where the pins are supported by a flexible conductive pad laminated with a non-conductive adhesive base. The pins are simply then inserted through the base ad held there, the cross-sectional view shown in FIG. 11. FIG. 12 simply illustrates such a pad made in a different shape, whilst FIG. 13 illustrates a single pad for two rows of pins. FIG. 14 illustrates an embodiment where the pins are fixed in position to the conductive pad.

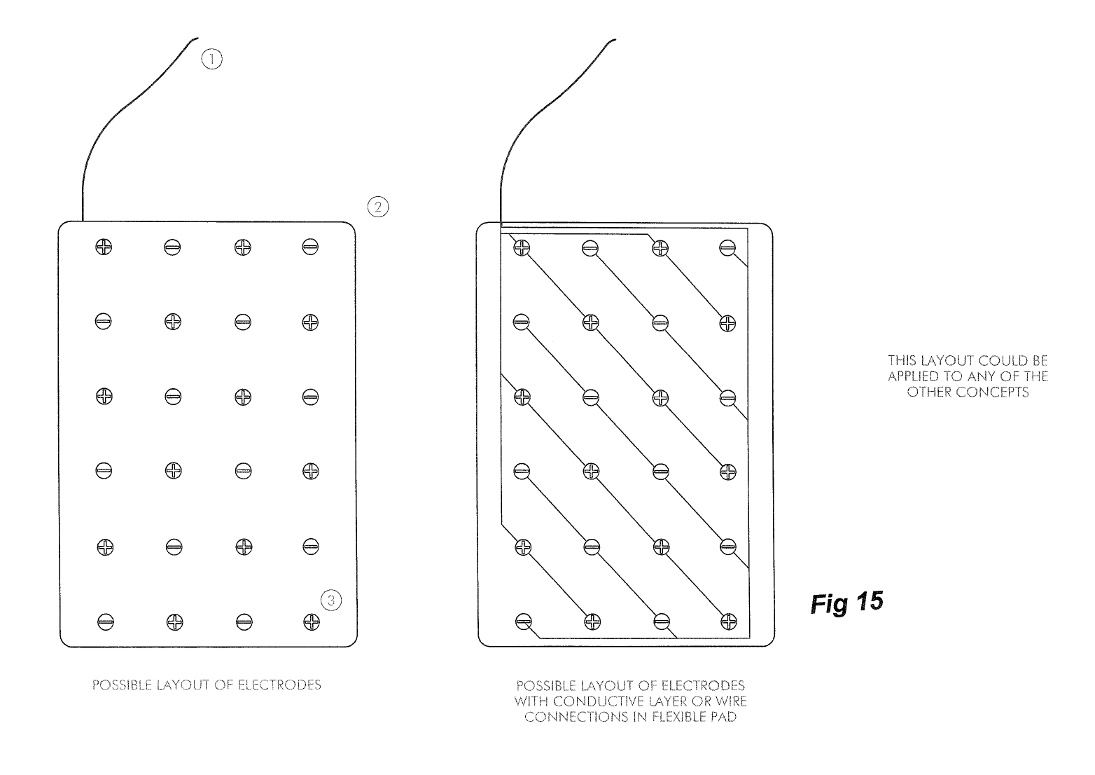

Illustrated in FIG. 15 is a possible layout of the electrodes with either a conductive layer or wire connections in a flexible pad.

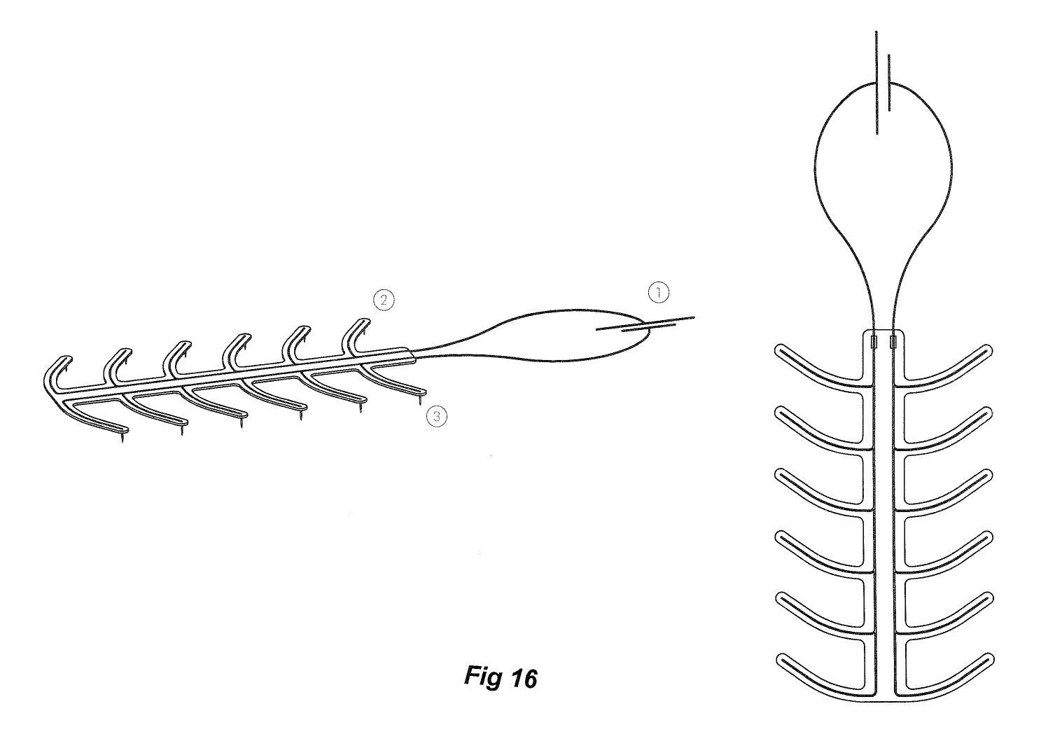

Finally illustrated in FIG. 16 is a single flexible PCB that connects two rows of electrical pins.

The reader should now appreciate the invention. Whilst the MNE has a flexible conductive stem, allowing it to be curved around the contours of the skin, it is not designed to allow the patient to have electrolipolysis treatment whilst ambulant for extended periods of time. Such an electrode design transmits mechanical forces from one needle to the next along the stem, effectively causing pain or dislodgement of the electrodes from the subcutaneous layer when there is movement of the patient.

The subcutaneous lipolysis electrode (FLE) that is described in this specification is designed to improve upon the MNE and allow long term electrolipolysis treatment to be carried out on a fully ambulant patient doing normal daily duties.

The invention is tailored to deliver electrolipolysis whilst the patient is ambulant and doing normal daily duties. This involves a miniaturised portable electrical apparatus carried by the patient to deliver electrical information to the new electrode invention referred to as the subcutaneous lipolysis electrode (SLE). This will allow the patient to carry out normal duties whilst having the electrolipolysis treatment. The treatment time can be comfortably increased in order to achieve greater amounts of lipolysis i.e., 1 to 12 hours instead of 1 hour. As a consequence, the treatment results improve several folds within a shorter time frame. Treatment results include a reduction of fat cell volume, anthropometric measurements, as well as an increase in biochemical indicators of lipolysis.

The SLE differs to the MNE in that the conductive stem is not only flexible, it can extend, conduct, contract and move so that any two needles attached to this stem float independently when placed in the subcutaneous layer without transmitting force to each other as they move with the body.

Increasing lipolysis by increasing treatment time gives electrolipolysis further opportunity in cosmetic medicine as a treatment for reducing greater volumes of localised fat bulk or cellulite in men and women. In the simplest description, SLE aims to transfer electrical information generated from an attached medical device to the subcutaneous tissue beneath the skin. The information is conducted along a stem that has two or more invasive conductive needle electrodes attached. These needles are attached so that electrical information is transmitted evenly through all needle electrodes within the subcutaneous layer.

The needle electrodes are pressed perpendicularly through the skin and can be engineered to penetrate to any required depth by adjusting the length of the needle. These needles electrodes may contain an adhesive tape or the like that will allow then to adhere onto the skin once inserted into the subcutaneous layer.

A unique feature of this SLE is the capacity of each of these electrically in series invasive electrodes to remain attached to the skin and to each other, whilst also allowing total independent mechanical movement of each of the invasive electrodes. In effect, the SLE claims it will allow the information generated by a medical device that attaches to the SLE to reach the subcutaneous tissue whilst allowing full ambulation of the subject wearing the electrodes.

The electrical stimulator in the broad sense will be small enough to be portable and used by a patient during normal daily activity. It can mimic the specifications of existing electrolipolysis generators i.e. 15 Hz, 2-‘4 mA or 15 OuA, rectangular wave and biphasic as an example only. It may also have capacity to produce frequency specific micro-currents for use in health or pain control. It could be single use or reusable.

The reader will now appreciate the advantage of the present invention that teaches a series of invasive electrodes on a single flexible, expandable conductive stem. The conductive stem allows stretching, compression and all other movements necessary so that movement of any needle on the SLE will not transmit enough mechanical force to affect the positioning of any of the other placed needle electrodes. In addition to this, the conducting stem will allow electrical information to reach each electrode. The number of electrodes on each conductive stem is two or more.

Said differently, the conductive stem between each invasive electrode is designed to allow full and independent articulation between invasive electrodes. This allows invasive electrodes to be attached and embedded into the skin and subcutaneous tissue, whilst subjects remains fully ambulant and are treated by an electronic medical device.

Each invasive electrode penetrates through the skin layer and can be of any length. The length of the needle governs the depth of penetration which can be modified if required.

The diameter of the needle electrodes can vary and the needle can be solid or hollow. Each electrode on the common conductive stem is in series and can conduct electrical information (from a medical device) and distribute this beneath the skin evenly (subcutaneous voltage map), or if required, on the skin simultaneously. Each SLE is either an anode or cathode. The SLE will allow electrical conductive coupling to a medical electronic device. The head of each invasive needle electrode can be insulated at the point of contact with the skin in order to eliminate short circuiting with the skin. Each of the invasive needle electrodes can have if required, adhesive tape that will hold the needle electrode in place on the skin during treatment. The medical device will be small enough for the patient to carry around unnoticed. The medical device will have programmable control of variables that will be necessary for a prescription treatment for electrolipolysis. The SLE will be packaged and be available sterile for easy application.

The present invention has immediate application in electolipolysis, pain control, lymphatic drainage and wound healing although other applications will become available. The SLE will remain securely embedded into the subcutaneous layer and remain securely in place during routine daily activity. The SLE will allow long and short durations of electrolipolysis treatment to be carried out whilst the patient is normally ambulant. The SLE quantitatively delivers more lipolysis than other prior are whilst a patient is ambulant. The SLE expands the clinical applications of electolipolysis for use in reducing larger localised fat deposit. The SLE can reduce cellulite more effectively than traditional electolipolysis using either acupuncture needle electrodes or MNE.

It is to be understood that independent pin or needle articulation will depend on materials used as well as material shape. The shapes and systems that can provide articulation and the materials can vary since materials can have a selection of elastomeric grades.

Further advantages and improvements may very well be made to the present invention without deviating from its scope. Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the summary of the invention, except where the context requires otherwise due to express language or necessary implication, the word “comprising” is used in the sense of “including”, i.e. the features specified may be associated with further features in various embodiments of the invention.

1. A conductive device for delivering an electrical current into subcutaneous tissue comprising:
a stem and two or more invasive electrodes configured to allow for independent articulation between the stem and one of the two or more invasive electrodes.

2. A conductive device according to claim 1 wherein the stem is conductive.

3. A conductive device according to claim 1 or claim 2 wherein the two or more invasive electrodes comprise insulation at the point of contact with the skin.

4. A conductive device for delivering an electrical current into subcutaneous tissue comprising:
a stem and two or more invasive electrodes configured to allow for independent articulation between the two or more invasive electrodes.

5. A conductive device as in any one of the above claims further including a top layer.

6. A method for delivering an electrical current to subject in need thereof, the method comprising the step of applying a conductive device according to any one of claims 1 to 3 to the subject; the method optionally comprising the step of using an adhesive to hold the conductive device in place on the skin to allow ambulation in the subject.

7. A method according to claim 4 wherein the electrical current is delivered for a period of more than 1 hour.
